(19) United States
(12) Patent Application Publication
(10) Pub. No.: US 2003/0228382 A1
(43) Pub. Date: Dec. 11, 2003

(54) Foot Fixer Kit

(75) Inventor: Ivar Edward Roth, Newport Beach, CA (US)

Correspondence Address: DR. ROTH'S FOOTCARE PRODUCTS LLC, 1012 BRIOSO DRIVE SUITE 105, COSTA MESA, CA, 92627, US

(73) Assignee: Dr. Ivar Edward Roth, Costa Mesa, CA (US)

(21) Appl. No.: 10/250,151

(22) Filed: Jun. 6, 2003

Related U.S. Application Data
(60) Provisional application No. 60/387,075, filed on Jun. 6, 2002.

(51) Int. Cl. A61K 35/78; A61K 31/315

(52) U.S. Cl. 424/731; 424/742; 424/744; 424/757; 424/769; 514/494

(57) ABSTRACT

A topical antifungal, antimicrobial and antibacterial formulation and kit and a method of use includes the steps of combining an oil extract of melaleuca alternifolia and an oil extract of eucalyptus australiana in each of four formulations wherein, the first one of the four formulations is prepared by further combining ethanol, ethoxylated castor oil, and zinc phenosulphonate; the second one of the four formulations is prepared by further combining petrolatum, ethoxylated castor oil, jojoba oil, aloe vera and lanolin, glyceryl stearate, and octyl stearate; the third one of the four formulations is prepared by further combining ethoxylated castor oil, at least one lauryl sulphate, coccabetaine, and coconut diethanolamine; and the fourth one of the four formulations is prepared by further combining petrolatum, jojoba oil, aloe vera and lanolin.

### BACKGROUND OF INVENTION

### FIELD OF THE INVENTION

This invention relates generally to topical products for the reduction and elimination of skin and nail fungal, microbial and bacterial infections and more particularly to an antifungal, antimicrobial and antibacterial formulation in a kit of products and its method of use to achieve significant medical benefit.

### SUMMARY OF INVENTION

The present invention teaches certain benefits in construction and use, which give rise to the objectives described below.

A topical antifungal, antimicobial and antibacterial formulation and kit and a method of use includes the steps of combining an oil extract of melaleuca alternifolia and an oil extract of eucalyptus australiana in each of four formulations wherein, the first one of the four formulations, a spray, is prepared by further combining ethanol, ethoxylated castor oil, and zinc phenosulphonate; the second one of the four formulations, a balm, is prepared by further combining petrolatum, ethoxylated castor oil, jojoba oil, aloe vera and lanolin, glyceryl stearate, octyl stearate; the third one of the four formulations, a wash, is prepared by further combining ethoxylated castor oil, at least one lauryl sulphate, coccabetaine, and coconut diethanolamine; and the fourth one of the four formulations, a gel, is prepared by further combining petrolatum, jojoba oil, aloe vera and lanolin.

Other features and advantages of the present invention will become apparent from the following more detailed description.

Test Methods and Results

Purpose of the Test: To test the antifungal, antimicrobial and antibacterial effectiveness and response time of the kit and its components in comparison to competing products. Also to test level of results in comparison to no treatment.

Methods: I tested the kit and its components on approximately one hundred patients in my office for over a year as it was being developed. We tested the products using two methods: on half of the patients, our product was used on one foot and the second foot was left untreated. For the other half of the test subjects, we used our product on one foot and a competing product on the second foot. The patients' results were monitored approximately every eight to ten weeks in their return visits to the office.

Foot Wash: It was found that used alone, the Foot Wash can help resolve mild cases of athlete's foot and substantially reduced the fungal, microbial and bacterial population on the skin and nails. The patients reported that on their untreated foot, the athlete's foot did not clear up at all, but the foot treated with the foot wash usually cleared up within one to two weeks.

Foot Spray: The Foot Spray is antifungal, antimicrobial, antibacterial and antiperspirant. I had my patients spray only one foot, leaving the other foot untreated. Within about one week, mild cases of athlete's foot went away on the treated foot and at the same time cases of smelly feet caused by, fungal, microbial and bacterial infestation were completely resolved, while the untreated foot saw no improvement. Additionally, the test subjects reported that the spray was an extremely effective antiperspirant that helped their feet feel fresh and remain dry for about eight hours.

Foot and Nail Gel: Patients using the gel found that within approximately three to four weeks, there was clearing of fungal, microbial and bacterial infections at the base of the nails on the treated foot. It takes many months for the nail to grow out completely clear, depending on how quickly the patient's nails grow and how bad the problem was. In severe cases, a significant reduction in the amount of fungus, microbes and bacteria was found, but the nails were not always completely clear by the end of the test period. These patients wanted to continue using the product because of the significant improvement the gel had caused.

Some patients found that their nails showed very good response for several months, but then the results slowed down. However, with perseverance and sufficient instructional compliance the fungal, microbial, bacterial issues will respond to the kit/nail gel and clear. As described previously, use of toe cot(s) at night is an excellent way to shorten the response time.

Patients with athlete's foot, cracked heels as well as fungal, microbial and bacterial infections of the nail and skin were instructed to also apply the gel to the affected areas on their test foot. They found that their athlete's foot cleared up within a week or two, and they had significant reduction in the number and severity of cracks in their heels and a significant reduction in the amount of fungus, microbes and bacteria on the skin were found.

Foot Balm: We noticed that when the balm was applied twice daily to the foot, moderate athlete's foot was cleared up within one to two weeks and the microbial and bacterial population on the skin and nails were substantially reduced. The patients reported that they had no improvement on their untreated foot.

Foot Fixer Kit: We also tested the products effectiveness by combining the treatments, using two of the products in combination. We noticed that when the patient used two or more products, the effectiveness increased significantly. Patients using the entire kit had the best results of all our test subjects, with significant improvement of their athlete's foot, cracked heels, infections of the nails and skin aused by fungal, microbial and bacterial infecstation.

Results: It was noted that our product had far superior results than the competing products. We found that competing topical medications had an effectiveness rate of 30 to 40%, and oral medications had an effectiveness rate of 75 to 80%. However, the kit had an effectiveness rate of 80% in treating fungal, microbial and bacterial infections of the nails, and 90% in treating athlete's foot (fungal) and microbial amd bacterial infections of the skin.

Another result of use of the kit that was not observed with other types of treatment is that the actual shape of the nail improved. Through the treatment program, thick, curved nails became thinner and less curved. We believe that it is in some part due to softening of the nail through the moisturizing quality of the gel. Additionally, loose nails that were not attached to the nail bed became attached with use of the kit.

Summary: In summary, mild athlete's foot is cleared up and fungal, mcrobial and bacterial populations on the skin are greatly reduced.wwen the W ashand the sSrSa whee eusd alone. With te Balm, a mnd gel oderatea thllte's fot is cleared up and fungoal, micrbial and bacterial populations on the skin and nails are greatly reduced. When you combine the use of all of these in the kit and use them regularly, practically every type of athlete's foot infection, microbial and bacterial infection ni the skin can be resolved within one or two weeks.

Mild fungal, microbial and bacterial infections aof the nail re cleared up with the gel alone while moderate and severe fungal, microbial and bacterial infections rof the nail esponds best to the complete kit. In both circumstances use of the toe cots at bedtime will enhance absorption and are highly recommended

Treatment time and results are dependent on patient instructional compliance and how quickly the nail grows.

While the invention has been described with reference to at least one preferred embodiment, it is to be clearly understood by those skilled in the art that the invention is not limited thereto. Rather, the scope of the invention is to be interpreted only in conjunction with the appended claims.

### DETAILED DESCRIPTION

The present invention is a topical antifungal, antimicrobial and antibacterial formulation comprising, in combination: an oil extract of melaleuca alternifolia, otherwise known as tea tree oil and an oil extract of eucalyptus australiana, otherwise known as eucalyptus oil. In the preferred embodiment, the two oils are mixed with carriers, emollients, surfactants, essential oils, fragrances and preservatives to prepare topical formulations that are easy to use and are acceptable in appearance and odor. The oils are combined with ethanol, ethoxylated castor oil, and zinc phenosulphonate to produce a spray. The oils are combined with petrolatum, ethoxylated castor oil, jojoba oil, aloe vera and lanolin, glyceryl stearate, and octyl stearate to produce a balm. The oils are combined with ethoxylated castor oil, at least one lauryl sulphate, coccabetaine, and coconut diethanolamine to produce a wash. Finally, the oils are combined with petrolatum, jojoba oil, aloe vera and lanolin to produce a gel. Each of these formulations when properly applied achieves certain objectives and all four of the formulations may be applied in combination to provide certain synergistic effects as will be described below. A topical antifungal, antimicrobial and antibacterial kit may be made up of the four formulations.

The invention further includes a method of treating fungal, microbial and bacterial infestations of the skin and nails. The kit is comprised of: the topical application of the formulations containing the two oils in combination with the other ingredients as defined above.

The purpose of the kit is to change the environment of the skin to combat fungal, microbial and bacterial infaections in the skin and nails on a continuing basis. Other topical antifungal, antimicrobial and antibacterialproducts only treat one aspect of the problem, and therefore offer a less complete and effective treatment. Oral antifungal medications, while having a success rate similar to the present formulations, are more expensive and may cause serious damage to the liver and to the heart, causing death in some cases. The unique combination of natural anti-ungal, antimicrobial and antibacterialingredients makes each of the formulations capable of non-toxic treatment of fungias, microbial and bacterial nfestation.s of the skin and nails Although each formula may be used individually, the combination provides maximum benefit.

The combination of the four formulations provides continuous antifungal, antimicrobial and antibacterial activity. The preferred application of the formulations includes placing a small amount of the wash on the effected and surrounding area of the skin before showering or bathing. The wash should be rubbed into the skin until fully absorbed. It will kill fungus, microbes and bacteria on contact. The nails should be scrubbed with a brush when using the wash during the shower. When the feet/hands have been dried the spray is applied to the bottoms of the feet/hands and between the toes/fingers. The gel is then applied twice daily to the nails. The effectiveness of the second gel application at bedtime can be enhanced with the use of a toe/finger cot. The toe/finger cot is slipped on the desired toe/finger after the application of the gel as a means of enhancing absorption. The petroleum substrate also holds the active ingredients for extended benefit.

The balm is applied to the rest of the foot/hand or surrounding skin area in the evening after applying the gel. It is rubbed into the skin surface until completely absorbed. The above describes a minimum treatment and may be repeated frequently for improved results.

The gel can be used independently, twice daily on mild cases of fungal microbeialand bacteria I infectionthat involves only one or two nails. As described previously, use of a toe/finger cot at night is highly recommended as a means of enhancing absorption and shortening the response time.

The several products in the kit described above are most advantageously and beneficially used in the order: wash, spray, gel and balm.

One of the key components of treatment is keeping the nails as short as possible and scrubbing the debris out of the nail with a nailbrush or using a nail file to carefully remove fungal, microbial and bacterial debris from under the nail. If the nails are completely involved, it may take a year or more to treat.

1. A topical antifungal, antimicrobial and antibacterial formulation comprising, in combination: an oil extract of melaleuca alternifolia and an oil extract of eucalyptus australiana.

2. The combination of claim 1 further comprising at least one of: carriers, emollients, surfactants, essential oils, fragrances and preservatives.

3. The combination of claim 2 further comprising ethanol, ethoxylated castor oil, and zinc phenosulphonate.

4. The combination of claim 2 further comprising petrolatum, ethoxylated castor oil, jojoba oil, aloe vera and lanolin, glyceryl stearate, and octyl stearate.

5. The combination of claim 2 further comprising ethoxylated castor oil, at least one lauryl sulphate, coccabetaine, and coconut diethanolamine.

6. The combination of claim 2 further comprising petrolatum, jojoba oil, aloe vera and lanolin.

7. A topical antifungal, antimicrobial and antibacterial kit comprising, in combination: an oil extract of melaleuca alternifolia and an oil extract of eucalyptus australiana in each of four formulations wherein the first one of the four formulations further comprises ethanol, ethoxylated castor oil, and zinc phenosulphonate; the second one of the four formulations further comprises ethanol, ethoxylated castor oil, and zinc phenosulphonate; the third one of the four formulations further comprises ethoxylated castor oil, at least one lauryl sulphate, coccabetaine, and coconut diethanolamine; and the fourth one of the four formulations further comprises petrolatum, jojoba oil, aloe vera and lanolin.

8. A method of treating fungal, microbial and bacterial foot infestation comprising the topical application of an oil extract of melaleuca alternifolia combined with an oil extract of eucalyptus australiana.

9. The method of claim 7 further comprising the step of further combining at least one of: carriers, emollients, surfactants, essential oils, fragrances and preservatives.

10. The method of claim 8 further comprising the step of combining ethanol, ethoxylated castor oil, and zinc phenosulphonate.

11. The method of claim 8 further comprising the step of combining petrolatum, ethoxylated castor oil, jojoba oil, aloe vera and lanolin, glyceryl stearate, and octyl stearate.

12. The method of claim 8 further comprising the step of combining ethoxylated castor oil, at least one lauryl sulphate, coccabetaine, and coconut diethanolamine.

13. The method of claim 8 further comprising the step of combining petrolatum, jojoba oil, aloe vera and lanolin.

14. A topical antifungal, antimicrobial and antibacterial method comprising the steps of combining an oil extract of melaleuca alternifolia and an oil extract of eucalyptus australiana in each of four formulations wherein, the first one of the four formulations is prepared by further combining ethanol, ethoxylated castor oil, and zinc phenosulphonate; the second one of the four formulations is prepared by further combining petrolatum, ethoxylated castor oil, jojoba oil, aloe vera and lanolin, glyceryl stearate, and octyl stearate; the third one of the four formulations is prepared by further combining ethoxylated castor oil, at least one lauryl sulphate, coccabetaine, and coconut diethanolamine; and the fourth one of the four formulations is prepared by further combining petrolatum, jojoba oil, aloe vera and lanolin.

15. A method of treating the feet using a wash, a spray, a gel and a balm comprising the steps of: placing a small amount of the wash on the feet before showering or bathing; rubbing the wash into the skin surface until fully absorbed; scrubbing toenails with a brush using the wash during a shower; applying a spray to the bottom of feet between toes thereof when the feet are dry; applying a gel twice daily to the toenails and the surrounding skin; enhancing effectiveness of the gel using a toe cot over the toes; applying a balm to the feet each evening after applying the gel; and rubbing the balm into the skin surface until completely absorbed.
